(19) United States
(12) Patent Application Publication
(10) Pub. No.: US 2025/0163358 A1
(43) Pub. Date: May 22, 2025

(54) MACHINE LEARNING ANALYSIS AND CONTROL OF CO2-FED PHOTOBIOREACTORS

(71) Applicant: Pacific AgriTec, LLC, Santa Maria, CA (US)

(72) Inventors: William B. Clary, Newburyport, MA (US); Ramon Elias, Santa Maria, CA (US)

(21) Appl. No.: 18/951,066

(22) Filed: Nov. 18, 2024

Related U.S. Application Data
(60) Provisional application No. 63/599,731, filed on Nov. 16, 2023.

(51) Int. Cl. C12M 1/36; C12M 1/00

(52) U.S. Cl. CPC C12M 41/48; C12M 21/02

(57) ABSTRACT

A system and method for predictive control of an algae reactor are disclosed. The method involves receiving correlation data from a correlation database, determining one or more correlations between parameters above a predetermined threshold, querying a parameter database to select associated data, training a machine-learning model based on the correlations and selected data, polling sensor data, predicting future values using the trained model, and instructing the algae reactor to perform required actions based on predicted values exceeding operational thresholds. The machine-learning model is trained to optimize predictions for improved control of the algae reactor.

### CROSS-REFERENCE TO RELATED APPLICATIONS

The present patent application claims the priority benefit of U.S. provisional patent application No. 63/599,731 filed Nov. 16, 2023, the disclosure of which is incorporated by reference herein.

### BACKGROUND OF THE DISCLOSURE

### Field of the Disclosure

The present disclosure is generally related to the capture of carbon dioxide produced from stationary sources or air by cultivating biomass such as microalgae which consumes carbon dioxide within a specially managed and controlled closed system that is at least partially controlled by a machine-learning model that optimizes various parameters.

### Description of the Related Art

Carbon dioxide (CO₂) is a greenhouse gas that is produced by mammal respiration, organic matter decomposition, fermentation processes, the combustion of fossil fuels, and other natural earth processes and sources. Separating and capturing carbon dioxide can add thirty percent (30%) or more to the cost of conventional stationary processes.

The added cost is due to the special apparatus, equipment and infrastructure that must be designed, built, and operated to separate, capture and transport this gas for other uses or for sequestration. These added costs pose a significant challenge, for industries or regions with limited financial capabilities not suited for adopting reusable energy sources such as wind and solar power which hinders the widespread adoption of carbon capture technologies.

Overall process efficiency losses are also incurred due to the need for additional energy to perform carbon capture and separation. An example are post-combustion CO₂ capture methods that use solvent-based capture techniques which require additional energy.

There are limited uses and places to permanently store captured CO₂. After the capture process, the carbon dioxide needs to be transported and used in other applications, or securely stored in adequate geological sites to prevent its release into the atmosphere. Establishing an extensive network of pipelines, storage facilities, and suitable underground geological formations for long-term storage requires substantial investments and careful planning which result in higher costs and logistical complexities. Suitable storage sites are often not located where the need exists.

Underground CO₂ storage can result in escape of the gas through pre-existing pathways such as wellbores and rock fissures, the contamination and displacement of groundwater, and cause seismic activity. Instances of public opposition to underground CO₂ storage have occurred and are likely to persist.

There is, therefore, a need in the art for improved systems and methods that improves the efficiency and effectiveness of carbon capture, utilization, and storage, while mitigating challenges associated with the same.

### SUMMARY OF THE CLAIMED INVENTION

Embodiments of the present invention include systems and methods for predictive control of an algae reactor. The method may involve receiving correlation data from a correlation database, determining one or more correlations between parameters above a predetermined threshold, querying a parameter database to select associated data, training a machine-learning model based on the correlations and selected data, polling sensor data, predicting future values using the trained model, and instructing the algae reactor to perform required actions based on predicted values exceeding operational thresholds. The machine-learning model is trained to optimize predictions for improved control of the algae reactor.

### BRIEF DESCRIPTION OF THE DRAWINGS

FIG. 1 illustrates an exemplary network environment in which a system for CO₂ capture and biomass-based storage may be implemented.

FIG. 2 illustrates a flowchart illustrating an exemplary method for data collection and correlation related to CO₂ collection and processing, algae reactor operation, and algae harvesting.

FIG. 3 illustrates illustrating an exemplary method for training a machine-learning model that predicts parameter values and triggers actions if predicted parameter values exceed set limits.

FIG. 4A illustrates an example parameter database.

FIG. 4B illustrates an example correlation database.

FIG. 5 illustrates a block diagram of an exemplary computing system that may be used to implement an embodiment of the present invention.

FIG. 6 illustrates an example neural network architecture, according to one aspect of the present disclosure.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

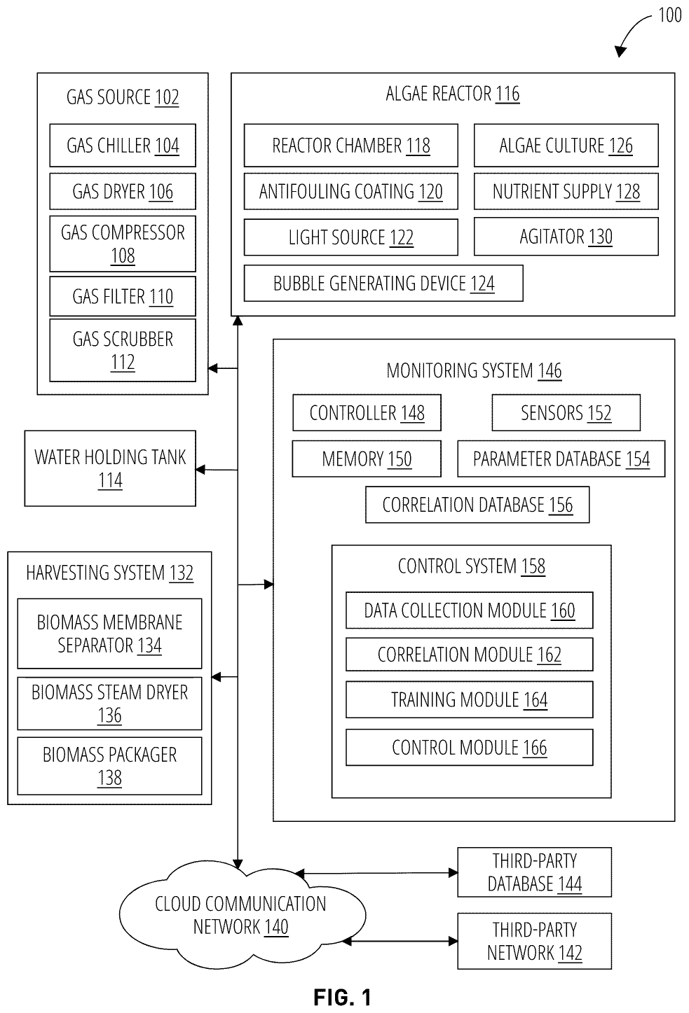

FIG. 1 illustrates a CO₂ fed photobioreactor control system. The CO₂ fed photobioreactor control system 100 may comprise a gas source 102, which may be any source of gas, particularly those containing CO₂, such as the atmosphere or point sources. Point sources of CO₂ may include industrial processes such as combustion processes, which involve the burning of fossil fuels for energy generation, heating, or other purposes. Combustion processes in power plants, refineries, and factories release CO₂ as a result of the chemical reaction between fuel and oxygen. Concrete production is another significant point source of CO₂ emissions. Cement, a key component of concrete, is produced through a process called calcination during which limestone (calcium carbonate) is heated to high temperatures, resulting in the release of CO₂ as a byproduct. Metallurgical processes such as iron and steel production release CO₂ as a byproduct of the reduction of iron ore using carbon (coke).

The gas source 102 may include a gas chiller 104 that directs gas to a heat exchanger, which may facilitate the transfer of heat from the gas to a cooling medium. The cooled gas may then enter a gas compressor 108, which may increase its pressure for subsequent cooling and liquefaction. The gas may then be directed through a series of condensers, expansion valves, and evaporators. As the gas flows through the gas chiller 104 and exchanges heat with a refrigerant which circulates through a closed loop, the refrigerant may absorb the heat from the gas, causing it to cool down. The specific design and configuration of the gas chiller 104 may depend on factors such as the volume and temperature of the gas, as well as the specific requirements of the application. Different cooling techniques, such as indirect cooling using refrigerants, are employed to achieve the desired cooling and liquefaction of the gas.

The gas source 102 may include a gas dryer 106 that removes water vapor from a gas, particularly a gas containing CO₂. In some cases, a gas dryer 106 may be a water dropout filter or a desiccant dryer. This type of dryer may typically consist of a vessel filled with a desiccant material, such as silica gel or activated alumina. The gas containing CO₂ may be directed into the dryer vessel, where it comes into contact with the desiccant. The desiccant material may have a high affinity for water molecules, so as the gas passes through the dryer, the desiccant adsorbs the moisture from the gas. This adsorption process effectively removes the water vapor, leading to a reduction in the humidity of the gas. The dried gas may then exit the gas dryer 106.

To maintain the efficiency of the drying process, desiccant dryers may include regeneration heaters or blowers, which periodically regenerate the desiccant material by heating the desiccant to remove the absorbed moisture, allowing it to be reused for subsequent drying cycles. The specific design and configuration of the gas dryer 106 may vary based on factors such as the flow rate and temperature of the gas, as well as the desired moisture content. Additionally, for larger-scale industrial applications, specialized drying systems can be employed, such as adsorption dryers or membrane dryers, which offer more advanced moisture removal capabilities.

The gas source 102 may include a gas compressor 108 consists of various stages or cylinders. Each cylinder contains a piston or impeller that oscillates, reducing the volume of the gas and increasing its pressure. As the piston or impeller moves, it compresses the gas within the cylinder. This compression process involves reducing the space available for the gas to occupy, causing its molecules to come closer together increasing the density and pressure of the gas. The gas is progressively compressed as it passes through multiple stages or cylinders, with each stage contributing to a further increase in pressure. Gas compressors 108 can be driven by various sources of power, such as electric motors or engines. Depending on the application, different types of compressors may be utilized, including reciprocating compressors, rotary screw compressors, or centrifugal compressors. The specific choice of compressor depends on factors such as the desired compression ratio, flow rate, and efficiency requirements. The compressed gas containing CO₂ can be utilized for various purposes, including storage, transportation, or further processing. Higher pressure enables more efficient storage or transport of the gas, reducing the required storage or transportation volume.

The gas source 102 may include a gas filter 110 removes particulates and other contaminants from a gas stream, such as a stream of gas containing CO₂. A gas filter 110 is comprised of one or more filters designed to capture undesired particles and contaminants present in the gas. The filters used may vary depending on the nature and size of the particles to be removed. Common filter types include particulate filters, bag filters, cartridge filters, electrostatic precipitators, etc. The gas filter 110 is designed with porous media that allows the gas to pass through while trapping the particulates and contaminants. These trapped particles can include dust, ash, soot, or other solid impurities present in the gas stream. The filtration process operates based on various mechanisms, such as mechanical sieving, interception, or diffusion.

Larger particles are typically captured by mechanical sieving, as they are unable to pass through the small openings in the filter media. Smaller particles may be removed through interception, where they collide with and adhere to the filter fibers. Diffusion captures ultrafine particles by causing them to collide and stick to the filter media due to Brownian motion. Regular maintenance and periodic replacement of filters may be necessary to ensure optimal filtration efficiency. Over time, the filters may become clogged with captured particles, reducing their effectiveness. By replacing or cleaning the filters, the filtration system can continue to efficiently remove particulates and contaminants from the gas stream. Removing particulates and contaminants helps protect downstream equipment and processes from damage or fouling.

The gas source 102 may include a gas scrubber 112 typically consists of a vessel or tower filled with a scrubbing solution, such as water or a chemical reagent. The gas to be treated is directed into the scrubber, where it comes into contact with the scrubbing solution. As the gas flows through the gas scrubber 112, chemical reactions occur between the contaminants in the gas and the scrubbing solution. These reactions result in the absorption or neutralization of the contaminant chemicals or components present in the gas. The scrubbing solution acts as a medium to capture or react with the contaminants, effectively removing them from the gas. The specific design of the gas scrubber 112 can vary based on the contaminants and chemicals present in the gas. Different types of gas scrubbers 112, such as wet scrubbers or dry scrubbers, may be utilized depending on the requirements of the gas treatment process. In addition to the scrubbing solution, the gas scrubber 112 may also incorporate additional components such as mist eliminators or demisters. These components help remove any liquid droplets or mist formed during the scrubbing process, ensuring that the treated gas leaving the gas scrubber 112 is as free from contaminants as possible. A gas scrubber 112 helps create a suitable and safe gas stream for algae cultivation. A water holding tank 114 is a storage vessel designed to hold water received from various sources such as wells, municipal sources, water treatment facilities, or as a byproduct of industrial processes like drying a CO₂-containing gas or separating water from an algal slurry.

The CO₂ fed photobioreactor control system 100 may include a water holding tank 114 is typically constructed using materials that are resistant to corrosion and contamination, such as concrete, steel, or plastic. The primary function of a water holding tank 114 is to store water for later use or to facilitate the management and distribution of water within a system such as an algae reactor 116. The water holding tank 114 is equipped with inlet and outlet connections, allowing water to enter and exit as needed. The inlet connection is connected to one or more water sources, while the outlet connection provides a means for drawing water from the tank. A water holding tank 114, may include various components such as level sensors or float switches to monitor and control the water level, pressure regulators to maintain consistent water pressure, and valves for controlling the flow of water in and out of the water holding tank 114.

The water holding tank 114 may operate based on the principle of maintaining a balance between water inflow and outflow. When water enters the water holding tank 114 from a source, it fills the water holding tank 114 until it reaches a predetermined level. Once the water holding tank 114 is full, the inflow is regulated, and the excess water may be redirected back to the source, to other parts of the system, or may be expelled to a drain or to the environment. In some embodiments, the water may be treated via UV light and/or one or more filters to kill and/or remove bacteria and other live organisms in the water. The water may be treated to remove metals and transition elements, such as by an ion exchange filter.

The CO₂ fed photobioreactor control system 100 may include an algae reactor 116, also known as an algal photobioreactor, is a specialized system designed to cultivate algae using a CO₂-containing gas, such as a CO₂-containing waste gas from an industrial process or combustion. The algae reactor comprises a reactor chamber 118 which allows light to penetrate a growth medium and supports the growth of algae. Within the reactor chamber 118, an algal slurry or suspension is maintained, which serves as the growth medium for the algae. The algae reactor may include a bubble generating device that augments gas lifting of the algae slurry. Algae harvesting may be done from weirs located at the top of the algae reactor with or without the use of coagulants or flocculants. The CO₂ containing waste gas may be fed through the bubble generating device 124 which evenly disperses the CO₂ containing gas into the algal slurry and may further use bubble lift processes to achieve fluid circulation and mixing while ensuring efficient contact between the CO₂ and the algae cells. This CO₂ containing gas serves as the carbon source for algae photosynthesis.

The algae reactor 116 may be equipped with one or more light sources 122, such as LED panels, strips, tubes, or fluorescent lamps, to provide the necessary illumination for photosynthesis. The intensity and duration of light exposure may be controlled to optimize algae growth and productivity. The algae reactor 116 may incorporate a nutrient supply 128 and/or delivery system to deliver solutions containing essential elements like nitrogen, phosphorus, and potassium which may be added to the algal slurry to support algae growth. The nutrient concentrations may be monitored and adjusted to maintain an optimal balance for algae cultivation. The algae reactor 116 may include a temperature regulation system to maintain the desired temperature range for optimal algae growth. This may be achieved through heating or cooling mechanisms, such as heat exchangers or temperature controllers.

The algae reactor 116 may include a reactor chamber 118 that provides a controlled environment for the cultivation of algae. The reactor chamber may be a sealed container or vessel and may be made of transparent material, such as glass or plastic, which may allow light to penetrate into the chamber. The algae reactor may also comprise metal materials, such as iron, steel, aluminum, etc. to facilitate heat transfer. The reactor chamber 118 may contain mixing mechanisms or agitator 130, such as stirrers or circulation pumps, to ensure even distribution of the algae and nutrients throughout the reactor chamber 118.

The reactor chamber 118 may incorporate a light source 122 to provide the necessary illumination for photosynthesis. Light-emitting diodes (LEDs) or fluorescent lamps are commonly used to deliver the specific wavelengths and intensities of light required for optimal algae growth. The light source is positioned in a way that evenly distributes light across the algal slurry, ensuring uniform exposure for all algae cells. The light source may be located within the reactor chamber 118.

The algae reactor 116 may include an antifouling coating 120 that may prevent the attachment and growth of unwanted organisms, such as biofilms, algae, or other fouling agents, on the surfaces of the algae reactor 116 components. The antifouling coating 120 may comprise a specialized material or chemical formulation that inhibits the adhesion and colonization of fouling organisms. The antifouling coating 120 may be applied to the surfaces of the reactor chamber 118, including the transparent vessel, gas delivery system, and other relevant components. In some embodiments, the surfaces contacting the algal slurry possess antifouling characteristics and may not require an antifouling coating 120. The antifouling coating 120 may comprise biocides or antimicrobial agents. These agents may be incorporated into the coating formulation and may release over time to deter the growth of microorganisms and algae. The biocides act by disrupting cellular processes or inhibiting the colonization of fouling organisms, thereby preventing their attachment and growth on the coated surfaces. The antifouling coating 120 may modify surface properties such as surface energy or roughness, to create an unfavorable environment for fouling organisms. For example, hydrophobic coatings can reduce the ability of waterborne organisms to adhere to the surface, making it more difficult for them to establish and grow.

The algae reactor 116 may include a light source 122 provides the necessary illumination for photosynthesis, supporting the growth and productivity of the algae. The light source 122 in an algae reactor may emit specific wavelengths of light that are optimal for photosynthetic activity. LED panels or fluorescent lamps are commonly used as light sources due to their energy efficiency and controllable light spectrum. These light sources 122 may be positioned around and/or within the reactor to ensure uniform distribution of light throughout the algal slurry. The light source 122 operates based on the principle of converting electrical energy into light energy.

In the case of LEDs, electric current passes through a semiconductor material, causing the release of photons that generate light. Fluorescent lamps work by passing an electric current through a gas-filled tube, which results in the excitation of mercury atoms and the subsequent emission of ultraviolet light. This ultraviolet light is then converted into visible light through a phosphor coating on the inner surface of the lamp. The intensity and duration of light exposure in the algae reactor may be controlled to optimize algae growth and productivity. Light intensity may be adjusted by controlling the power supply or dimming capabilities of the light source and cycling the light for certain periods and durations either with a sign wave or square wave profile. Additionally, timers or programmable controllers may be used to regulate the duration and timing of light exposure, simulating day and night cycles to maintain proper growth conditions for the algae or in an alternate pattern and/or frequency to optimize algae growth.

The algae reactor 116 may include a bubble generating device 124 that facilitates efficient gas-liquid contact between the CO₂-containing waste gas and the algal slurry by dispersing bubbles of the gas within the algae culture 126 to enhance the transfer of CO₂ to the algae cells. The bubble generating device 124 may additionally act to cool the liquid via the injection of cool bubbles. The bubble generating device 124 typically comprises of a micro-bubbler, diffuser, or membrane, which produces fine bubbles.

A CO₂ containing gas may flow through the micro-bubbler encountering small pores or perforations in the material, causing the gas to be released in the form of numerous small bubbles. These bubbles rise to the surface of the algal slurry, creating a gas-liquid interface where the CO₂ can be efficiently absorbed by the algae cells. In some embodiments, at least some of the CO₂ is solubilized into the water of the algal slurry. The rising action of the bubbles may additionally perform the function of agitating the algae. The bubble generating device 124 may include a distribution system to ensure even dispersion of the bubbles throughout the algal slurry.

The algae reactor 116 may include an algae culture 126 that is a population of algae cells which are prepared for growth within an algae reactor 116. The algae culture 126 is typically maintained as an algal slurry or suspension. The slurry contains water, nutrients, and the algae cells. The water serves as the medium for supporting algae growth, providing the necessary environment for their survival and reproduction. The algae culture 126 may be processed to remove microorganisms, such as via use of UV light, and other harmful elements, which may inhibit algae growth or compromise the efficiency of algae growth in an algae reactor 116.

The algae reactor 116 may include a nutrient supply 128 provides the necessary elements for the growth and metabolic processes of the algae culture 126. The nutrient supply 128 may comprise a controlled delivery of essential nutrients to the algal slurry which may include nitrogen (N), phosphorus (P), potassium (K), and other trace elements. The nutrient supply 128 may consist of nutrient solutions and/or additives that contain predetermined concentrations of nutrients. The nutrient supply 128 may include reservoirs or tanks that hold the nutrient solutions, dosing pumps and/or injectors that deliver the nutrients into the algal slurry, and monitoring devices to ensure control of nutrient concentrations. The dosing pumps and/or injectors may be programmed to deliver the nutrients at specific intervals or according to a predetermined schedule.

The nutrient supply 128 system may additionally incorporate sensors to monitor and adjust nutrient levels. An example of a nutrient supply 128 may include a pH supply and/or delivery system to deliver solutions containing essential ions and buffers to maintain a pH level which may be added to the algal slurry to support algae growth. In an embodiment, a nutrient supply may comprise a chelating supply and/or delivery system to deliver solutions containing essential chemical components and ions to reduce the metals and nutrients from the algae cell walls during harvesting and to support algae growth.

The algae reactor 116 may include an agitator 130 mixes and circulates the algal slurry, facilitating an even distribution of algae throughout the growth medium. The agitator 130 may comprise a motor or drive unit, which provides the necessary rotational force for the agitator. The motor may be connected directly, or indirectly via a shaft to one or more impellers or blades. The impellers or blades create turbulence and induce fluid motion within the algae reactor, ensuring uniform distribution of nutrients, gases, and light exposure to the algae culture 126. The shape and arrangement of the impellers may vary. The agitator 130 helps distribute nutrients evenly, ensuring uniform growth and preventing nutrient depletion or buildup in certain areas of the reactor. In some embodiments, the agitator 130 may not comprise a physical mechanism, but instead the effect may be achieved via the rising action of bubbles of CO₂ containing gas through the algal slurry.

The CO₂ fed photobioreactor control system 100 may include a harvesting system 132 that separates the algal biomass from the liquid medium and may utilize mechanical centrifuge, membranes, settling zones, a tank consisting of a conical floor, or baffles to increase the concentration of the algae cells. The algal slurry may be allowed to rest or undergo gentle agitation, which promotes the settling of algae cells to the bottom of the vessel. The settling of algae cells may occur within the algae reactor configured as discussed or may be transferred to a separate vessel for the same purpose. As the algae cells settle, a concentrated layer of biomass forms, while the liquid phase (supernatant) containing water and other components is decanted or drained off.

The concentrated algal biomass may be further dewatered using techniques such as filtration, centrifugation, or flocculation. Filtration involves passing the algal slurry through a filter medium that retains the algae cells while allowing the liquid to pass through. Centrifugation utilizes centrifugal force to separate the denser algae cells from the liquid phase. Flocculation involves the addition of chemical agents to induce the clumping of algae cells, facilitating their precipitation and separation from the liquid. In an ideal embodiment, the algal slurry is removed from the algae reactor 116 and most of the water is removed via the use of a biomass membrane separator 134. The water content of the algae may further be reduced via the use of a biomass steam dryer 136.

The harvesting system 132 may include a biomass membrane separator 134 that comprises a semi-permeable membrane having specific pore sizes that allow for the passage of liquid, dissolved substances, and smaller molecules while retaining the larger algae cells and biomass. The liquid phase, which contains water and smaller dissolved molecules, passes through the membrane and the water may be recovered and stored in a water holding tank 114. Via a combination of size exclusion and molecular sieving the algae cells, which are larger in size, are unable to pass through the small pores or openings of the membrane, while allowing the liquid components can permeate through the membrane structure due to their smaller size. The biomass membrane separator 134 may incorporate additional mechanisms such as backwashing or crossflow filtration. Backwashing involves periodically reversing the flow direction across the membrane surface to dislodge and remove accumulated particles and debris. Crossflow filtration utilizes a tangential flow of liquid across the membrane surface, preventing the buildup of fouling materials and maintaining optimal filtration performance.

The harvesting system 132 may include a biomass steam dryer 136 that comprises a drying chamber or vessel, a steam generator, and a heat exchanger. The harvested algal biomass is introduced into the drying chamber, which is designed to facilitate the drying process. The steam generator generates high-pressure steam, which raises the temperature within the drying chamber, facilitating the evaporation of moisture from the algal biomass. As the algal biomass is exposed to the heated environment, the moisture within the cells begins to vaporize and then condenses on the cooler surfaces within the drying chamber, such as condensation plates or heat exchange coils before being collected and removed from the system. The drying process continues until the desired moisture content is achieved, resulting in a dry and concentrated algal biomass. The dried biomass can then be further processed or utilized for various applications.

The harvesting system 132 may include a biomass packager 138 comprises a packaging machine or system, which may be automated or semi-automated and may include a conveyor belt, feeding mechanism, weighing system, and sealing apparatus The harvested algal biomass is fed into the biomass packager 138 by a conveyor belt or feeding mechanism. The algae is weighed to determine the desired packaging weight or quantity and is then deposited into the packaging material, such as bags or containers. The biomass packager 138 seals the package using any of heat sealing, adhesive sealing, or other mechanisms to ensure proper closure and preservation of the algal biomass. The packaging may comprise any of bulk packaging, individual bags, or containers of varying sizes.

The CO₂ fed photobioreactor control system 100 may include a cloud communication network 140 that is comprised of a network of remote servers that are interconnected and provide various services and resources over the internet. The cloud communication network 140 may include network infrastructure, servers, storage systems, and software applications. The network infrastructure allows for seamless connectivity and communication between the algae reactor and the cloud communication network 140 servers. The servers host the necessary software applications and algorithms required for data processing, analysis, and control. The storage systems provide secure and scalable storage capacity for data generated by the algae reactor. The cloud communication network 140 may facilitate remote control and management of an algae reactor. Operators may access a cloud-based control panel or dashboard to monitor and adjust various parameters of the reactor, such as CO₂ flow rate, nutrient, pH, chelating agent concentration, algae density, temperature, electricity consumption, and lighting conditions. The cloud communication network 140 also enables the storage and retrieval of historical data, analysis, and future optimization of the algae reactor's 116 performance.

The CO₂ fed photobioreactor control system 100 may include a third-party network 142 is an external network or platform that is utilized to enable functionality and capabilities of the algae reactor 116. A third-party network 142 may offer various services, data analysis tools, and/or additional data or resources to optimize the algae cultivation process and improve CO₂ utilization. A third-party network 142 may include a cloud-based platform, data analytics tools, and external APIs (Application Programming Interfaces). Data analytics tools within the third-party network 142 may enable advanced data processing and insights generation.

These tools may employ algorithms and machine-learning techniques to analyze the data collected from the algae reactor to provide valuable insights into such as CO₂ consumption rates, optimal nutrient dosage, growth patterns, and other performance indicators. These insights can be used to optimize the operation of the algae reactor 116, improve CO₂ utilization efficiency, and improve overall productivity. Furthermore, the third-party network 142 may offer external APIs that allow seamless integration between the algae reactor and external systems or services. These APIs enable data exchange and interoperability, facilitating collaborations with environmental monitoring agencies and/or industry partners including validation of carbon collected and stored within the produced algal biomass.

The CO₂ fed photobioreactor control system 100 may include a third-party database 144 that is any external database which may store data including operational parameters, environmental conditions, growth metrics, and historical records relating to an algae reactor. The third-party database 144 may store data related to growth characteristics of an algae culture 126 created by a vendor who prepares algae cultures 126. Similarly, a third-party database 144 may comprise manufacturer information relating to any component of an algae reactor 116 including gas collection and biomass harvesting. In some cases, the predetermined correlation threshold may be adjusted or calibrated based on the data from the third-party databases 144.

The CO₂ fed photobioreactor control system 100 may include monitoring system 146 for the algae reactor 116 that comprises at least a controller 148, memory 150, and sensors 152 within the algae reactor 116 which measure parameters such as CO₂ concentration, temperature, pH level, dissolved oxygen, nutrient levels, etc. The sensors 152 may continuously collect data, providing real-time information about the status of the algae reactor 116. The collected data from the sensors 152 may be transmitted to a central control unit or system. The control unit processes and analyzes the data and may modify one or more parameters of an algae reactor 116 in response to the data collected from one or more sensors 152. For example, the monitoring system 146 may control the flow rate of the CO₂-containing waste gas, adjust the lighting intensity and duration, regulate the nutrient dosing, monitor the overall algae reactor 116 conditions, etc.

The control unit may further communicate with a user interface which displays sensor 152 readings and other relevant information. The monitoring and monitoring system 146 may additionally or alternatively comprise a brokerage and/or arbitration function. For example, the monitoring and monitoring system 146 may determine the amount of CO₂ captured by harvested algal biomass, and may further determine a carbon credit value and/or facilitate the sale of the algal biomass, carbon credits, process byproducts, or other products and services related to an algae reactor 116 including equipment and/or licensing, maintenance and/or operational services, etc.

The monitoring system 146 may include the controller 148 that is a computing device comprised of a processor for performing computations and communicates with a memory 150 for storing data. The controller 148 may be in communication with a communications interface and/or one or more sensors 152 and may further be allowed to control the at least one function or process related to an algae reactor 116 and/or the brokerage and/or arbitration of products and/or services related to an algae reactor 116. The controller 148 may be a commercially available central processing unit (CPU) or graphical processing unit (GPU) or may be a proprietary, purpose-built design. More than one controller 148 may operate in tandem and may be of different types, such as a CPU and a GPU. A GPU is not restricted to only processing graphics or image data and may be used for other computations.

The monitoring system 146 may include memory 150 that serves as the electronic circuitry within a computing device that temporarily stores data for usage by the controller 148. The memory 150 may additionally comprise persistent data storage for storing data used by the controller 148. The memory 150 may be integrated into a controller 148 or may be a discrete component. The memory 150 may be integrated into a circuit, such as soldered on component of a single board computer (SBC) or may a removable component such as a discrete dynamic random-access memory (DRAM) stick, secure digital (SD) card, flash drive, solid state drive (SSD), magnetic hard disk drive (SSD), etc. In some embodiments, memory 150 may be part of a controller 148. Multiple types of memory 150 may be used.

The monitoring system 146 may include sensors 152 that collect data by measuring one or more physical properties such as CO₂ concentration, temperature, pH level, dissolved oxygen, and nutrient levels, etc. CO₂ sensors 152 are essential for measuring the concentration of CO₂ in the algae reactor 116 and may utilize infrared or electrochemical technology to detect and quantify the CO₂ concentration in the gas phase by measuring the absorption or electrical properties of a gas sample and converting it into a CO₂ concentration value. Temperature sensors 152 may comprise thermocouples, resistance temperature detectors (RTDs), optical temperature sensors, etc. pH sensors 152 monitor the acidity or alkalinity of an algal slurry and may utilize pH-sensitive electrodes and/or indicators that generate electrical signals corresponding to the hydrogen ion concentration in the liquid medium. Nutrient sensors 152 may monitor the levels of essential nutrients such as nitrogen, phosphorus, and potassium within the algae reactor 116.

The monitoring system 146 may include the parameter database 154 that stores data collected by one or more sensors 152 measuring one or more parameters related to the collection and/or processing of CO₂, the operation of an algae reactor 116, and/or the harvest and processing of algae. Parameters may include any of the concentration of CO₂ and/or one or more component gases in a CO₂ containing gas from a gas source 102 and/or at any point during processing of the CO₂ containing gas. Additional parameters may include moisture content, pH, temperature, pressure, etc. Parameters relating to the operation of an algae reactor 116 may include any of energy usage, light intensity and/or penetration, CO₂ concentration in solution, concentration of one or more nutrients such as nitrogen, potassium, phosphates, etc. Additional relevant parameters may include the concentration of algae, pH, temperature, agitation and/or movement of algae resulting from an agitator 130 or the lifting action from a bubble generating device 124, etc. Parameters relating to the harvesting of algae may include the water content of harvested algae, weight, temperature, etc.

The monitoring system 146 may include the correlation database 156 that stores correlation data comprising at least a first parameter, a second parameter, and a correlation coefficient. The correlation coefficient quantifies the relationship between the first parameter and the second parameter such that a higher correlation coefficient indicates a stronger relationship. A higher correlation coefficient, then the greater the change in the value of a second parameter for any change in the value of a first parameter.

The monitoring system 146 may include the control system 158 that initiates a data collection module 160 and receives data collected from one or more sensors 152 measuring one or more parameters related to the collection and/or processing of CO₂, the operation of an algae reactor 116, and/or the harvest and processing of algae. The collected sensor 152 data is sent to the correlation module 162 which calculates correlation coefficients for pairs of parameters. The correlation data is received and sent to a training module 164 which determines which of the pairs of parameters represent a significant correlation, selecting the significant correlations as features, and using the correlation and parameter data from the correlation database 156 and the parameter database 154 to train one or more machine-learning models related to the collection and/or processing of CO₂, the operation of an algae reactor 116, and/or the harvest and processing of algae.

The one or more machine-learning models are received and sent to the control module 166 which monitors one or more sensors and predicts future values of one or more parameters to determine whether the future values of the one or more values require action to be taken to maintain and/or optimize the operation of one or more of the collection and/or processing of CO₂, the operation of an algae reactor 116, and/or the harvest and processing of algae. Action is taken as necessary based upon the predicted future values of the one or more parameters and a reactor status is received. If the monitoring and control process is complete, then ending the process, otherwise repeating the data collection module 160, correlation module 162, training module 164, and control modules 166.

The monitoring system 146 may include the data collection module 160 that receives a data collection request from the control system 158 and initializes one or more sensors 152 measuring one or more parameters related to the collection and/or processing of CO₂, the operation of an algae reactor 116, and/or the harvest and processing of algae. The one or more sensors 152 are then polled and the collected data is saved to a parameter database 154 and sent to the control system 158. The correlation module 162 receives collected data from the control system 158 and may further receive data from a parameter database 154 and/or a third-party database 144. A first parameter and a second parameter are selected, and a correlation coefficient is calculated for the first parameter and the second parameter. The correlation data comprising at least the first parameter, second parameter, and correlation coefficient is saved to the correlation database and the process is repeated for all permutations of first and second parameters in the received parameter data.

The calculated correlation data may be sent to the control system 158. The training module 164 receives correlation data from the control system 158 and a correlation database 156 and selects a first set of correlation data comprising at least a first parameter, second parameter, and correlation coefficient. The correlation coefficient is compared to a predetermined threshold, and the correlation is selected as a feature if the correlation coefficient is equal to or greater than the threshold value. Each set of correlation data is compared to the predetermined threshold until none remains.

The parameter database 154 is then queried for data relating to the correlations selected as features, and the correlation and parameter data are used to train one or more machine-learning models. The trained machine-learning models are sent to the control system 158. The control module 166 receives one or more machine-learning models from the control system 158 and polls one or more sensors 152 measuring one or more parameters related to the collection and/or processing of CO₂, the operation of an algae reactor 116, and/or the harvest and processing of algae. The control module 166 may alternatively or additionally receive data from the control system 158 and/or the parameter database 154.

The one or more machine-learning modules predict the future value of one or more parameters related to the collection and/or processing of CO₂, the operation of an algae reactor 116, and/or the harvest and processing of algae. It is determined whether the predicted value indicates that action is required to maintain one or more of the collection and/or processing of CO₂, the operation of an algae reactor 116, and/or the harvest and processing of algae within operational or further, optimal, ranges, and performing the one or more actions as necessary. The required actions may be saved to a database and/or be configured by the user of a CO₂ fed photobioreactor monitoring and control system. A reactor status is then sent to the control system 158.

In some cases, a process for monitoring and controlling the operation of an algae reactor may begin with initiating the data collection module 160. The data collection module 160 may receives a request to collect data from one or more sensors 152. The process may continue with receiving collected data from the data collection module 160. The collected data may comprise measurements from one or more sensors 152 measuring at least one parameter related to the collection and/or processing of a CO₂ containing gas, the operation of an algae reactor 116, and/or the harvest and processing of algae.

The data collection module 160 may receive collected data, query a parameter database 154, and optionally query a third-party database 144 for data relating to one or more of the collection and/or processing of a CO₂ containing gas, the operation of an algae reactor 116, and/or the harvest and processing of algae. For the received data, selecting a first parameter and a second parameter and calculating a correlation coefficient using the data for both parameters. The calculated correlation data is saved to the correlation database 156. If there are additional second parameters, then another second parameter is chosen, otherwise if there are additional first parameters, then another first parameter is chosen until no additional first parameters and/or second parameters which have not had a correlation coefficient calculated remain. Correlation data may be received from the correlation module 162. In an embodiment, the correlation data may comprise at least a first parameter, a second parameter, and a correlation coefficient.

The training module 164 may be initialized and query the correlation database 156 and receives correlation data from which it receives a set of correlation data comprising a first parameter, a second parameter, and a correlation coefficient. If the correlation coefficient is above a significance threshold, the correlation data is selected as a feature. If there is more correlation data which has not been compared to the predetermined correlation threshold, then select another set of correlation data to be compared to the predetermined correlation threshold. When all correlation data has been compared to the predetermined correlation threshold, query the parameter database 154 for data related to the correlation data selected as features. Utilizing the correlation data selected as features and related parameter data received from the parameter database 154 to train a machine-learning model. A trained machine-learning module may be received from the training module 164.

The control module 166 may be initialized and receive a machine-learning model, poll one or more sensors 152, and receive at least one prediction for the future value of a parameter relating to one or more of the collection and/or processing of CO₂, the operation of an algae reactor 116, and/or the harvest and processing of algae. If the prediction requires action, such as if the predicted value is outside the operational parameters of the related process, such as a temperature outside the operational range for an algae reactor 116, then performing the required action.

A reactor status may be received from the control module 166. The reactor status may comprise a general status of nominal or anomalous operation or may comprise details such as parameter values and actions taken if necessary. Whether the monitoring and control process is complete may be determined. The monitoring and control process may be complete if the reactor status indicates that the algae has been harvested and processed and the algae reactor is no longer growing algae.

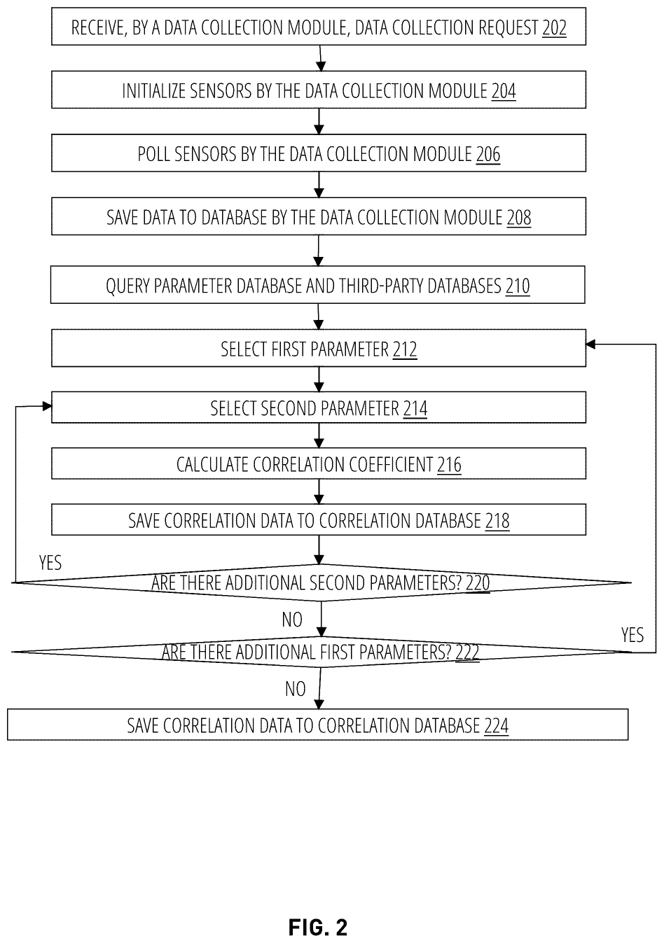

FIG. 2 illustrates a flowchart illustrating an exemplary method for data collection and correlation related to CO₂ collection and processing, algae reactor operation, and algae harvesting. The method of FIG. 2 may be performed based on execution of a data collection module 160 and a correlation module 162 by a processor in accordance with initiation and call by the control system 158.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

At step 202, the data collection module 160 may request to collect data from the control system 158. At step 204 the data collection module 160 may initialize one or more sensors 152. The one or more sensors 152 may measure at least one parameter related to the collection and/or processing of CO₂, the operation of an algae reactor 116, and/or the harvest and processing of algae. Initializing sensors 152 may comprise a handshake process where a message is sent to a first sensor 152 by the controller 148 of a monitoring and monitoring system 146, and a response is received from the at least one sensor 152 indicating that the message was received. The response may further indicate that the sensor 152 is operating properly. Initializing sensors 152 may additionally comprise calibration, which may be performed by comparing measurements from two or more similar sensors 152 and/or exposing the one or more sensors 152 to a condition with known parameters and comparing the measurements from the one or more sensors 152 to the value of the known parameter. Calibration may indicate whether a sensor 152 is operating properly and may additionally provide a correction factor to compensate for any identified deviations.

At step 206, the data collection module 160 may poll one or more sensors 152 measuring at least one parameter related to the collection and/or processing of CO₂, the operation of an algae reactor 116, and/or the harvest and processing of algae. Parameters relating to the collection and/or processing of CO₂ may include those relevant to one or more of a gas chiller 104, gas dryer 106, gas compressor 108, gas filter 110, and/or gas scrubber 112. Relevant parameters may include concentrations of CO₂ and/or one or more component gases received from a gas source 102 and/or at any point during processing of the CO₂ containing gas. Additional relevant parameters may include moisture content, pH, temperature, pressure, etc. Parameters relating to the operation of an algae reactor 116 may include any of energy usage, light intensity and/or penetration, CO₂ concentration in solution, concentration of one or more nutrients such as nitrogen, potassium, phosphates, etc. Additional relevant parameters may include the concentration of algae, pH, temperature, agitation and/or movement of algae resulting from an agitator 130 or the lifting action from a bubble generating device 124, etc. Parameters relating to the harvesting of algae may include the water content of harvested algae, weight, temperature, etc. In an embodiment, measuring a concentration of CO₂ from a gas source 102 at 20% and the moisture content at 15%. In another embodiment, measuring a temperature within an algae reactor 116 of 106. CO₂ fed photobioreactor control system 100° F. and a pH of 6.4. At step 208, the data collection module 160 may save the data collected from one or more sensors 152 to the parameter database 154. In some cases, the data collection module 160 may send the data collected from one or more sensors 152 to the control system 158.

The method may continue with receiving data collected from one or more sensors 152 from the control system 158. At step 210, the correlation module 162 may query the parameter database 154 for data collected from one or more sensors 152. The data may be real time data, or may be historical data collected from one or more algae reactors 116 and/or one or sensors 152 monitoring any of a CO₂ collection and/or processing apparatus or process, operation of an algae reactor, harvest and processing of algae, etc. At step 210, the correlation module 162 may also query one or more third-party databases 144 for data related to any of a CO₂ collection and/or processing apparatus or process, operation of an algae reactor, harvest and processing of algae, and/or any relevant equipment, processes, etc. In some cases, a third-party database 144 queried may comprise a manufacturer's database describing the operational parameters of a gas chiller 104, gas dryer 106, gas compressor 108, gas filter 110, gas scrubber 112, water holding tank 114, algae reactor 116, light source 122, bubble generating device 124, nutrient supply 128, agitator 130, harvesting system 132, etc.

In step 212, the correlation module 162 may select a first parameter relating to any of a CO₂ collection and/or processing of CO₂, operation of an algae reactor, or harvest and processing of algae. In an embodiment, the first parameter is the temperature of an algal slurry in the algae reactor 116. In some cases, the first parameter is the penetration of light through an algal slurry.

In step 214, the correlation module 162 may select a second parameter relating to any of a CO₂ collection and/or processing of CO₂, operation of an algae reactor, or harvest and processing of algae. In an embodiment, the second parameter is the pH of an algal slurry in the algae reactor 116. In another embodiment, the second parameter is the concentration of nitrogen in the algal slurry. In a further embodiment, the second parameter is the concentration of CO₂ in the algal slurry.

In step 216, the correlation module 162 may calculate a correlation coefficient which represents the relationship between the first parameter and the second parameter. The higher the correlation coefficient, the closer the relationship between the parameters, or more specifically, the more likely that a first parameter influences or is influenced by a second parameter. In an embodiment, calculating a correlation coefficient may comprise the least squares linear regression where a linear regression model or line is fit to the data set comprised of data where each data point comprises values representing each a first parameter and a second parameter. The slope of the model or line represents the change in the second parameter given a per unit change in the first parameter. The correlation coefficient is the square root of the coefficient of determination, which is the proportion of the total variance in the second variable as described by the line. In an alternate embodiment, the correlation coefficient may be the Spearman rank correlation coefficient.

In step 218, the correlation module 162 may save the calculated correlation data to the correlation database 156. Ins decision block 220, the correlation module 162 may determine whether there are additional second parameters. Additional second parameters may be any parameters which are not the same as the first parameter which have not had correlation coefficients calculated for the first parameter and the second parameter. In an embodiment, there is an additional second parameter, and therefore return to step 214 and select another second parameter. In decision block 222, the correlation module 162 may determine whether there are additional first parameters. Additional first parameters may be any parameters which have not had correlation coefficients calculated for one or more second parameters which are different than the first parameter. In an embodiment, there is an additional first parameter, and therefore return to step 212 and select another first parameter. In step 224, the correlation module 162 may send the correlation data to the correlation database 156.

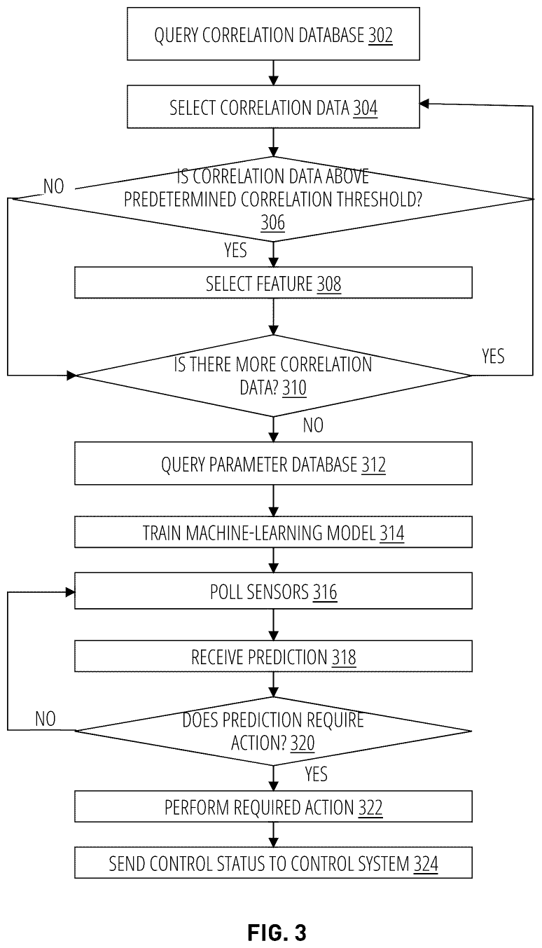

FIG. 3 illustrates illustrating an exemplary method for training a machine-learning model that predicts parameter values and triggers actions if predicted parameter values exceed set limits. The method of FIG. 3 may be performed based on execution of a training module 164 and a control module 166 by a processor in accordance with initiation and call by a control system 158.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The training module 164 may first receive correlation data from the control system 158. The correlation data comprises at least a first parameter, a second parameter, and a correlation coefficient. At step 302, the training module 164 may query the correlation database 156 for correlation data comprising at least a first parameter, a second parameter, and a correlation coefficient. In some embodiments, the data received from the correlation database 156 may be different than the correlation data received from the control system 158, such as correlation data previously calculated.

At step 304, the training module 164 may select correlation data from the received correlation data. The selected correlation data comprises a first parameter, a second parameter, and a correlation coefficient. At decision block 306, the training module 164 may determine whether the selected correlation is above a predetermined correlation threshold. In an ideal environment, the predetermined correlation threshold is 0.95, or 95%, therefore if the correlation coefficient is above 0.95, then the correlation data is above the predetermined correlation threshold. In some embodiments, the predetermined correlation threshold may be lower, such as 0.75. In some embodiments, a correlation coefficient which is equal to the predetermined correlation threshold may be considered to be above the predetermined correlation threshold. In an embodiment, the predetermined correlation threshold is 0.95 and the correlation coefficient is 0.98, therefore the correlation data is above the predetermined correlation threshold. In another embodiment, the correlation coefficient is 0.15, and therefore the correlation data is below the predetermined correlation threshold.

At step 308, the training module 164 may select the correlation data as a feature if the correlation coefficient is above the significance threshold. A feature is measurable property, characteristic, or phenomenon, which can be used to quantify relationships and patterns which can be recognized and utilized by machine-learning models. Features with significant relationships improve the accuracy and relevance of machine-learning models. At decision block 310, the training module 164 may determine whether there is more correlation data. There is more correlation data if there are more sets of parameters and correlation coefficients which have not been compared to the significance threshold. If there is more correlation data, then return to step 304 and select additional correlation data comprising a first parameter, second parameter, and correlation coefficient. At step 312, the training module 164 may query the parameter database 154 for parameter data related to the selected features. For example, if a correlation between algal slurry temperature and pH in an algae reactor 116 is selected as a feature, then receiving temperature and pH data.

At step 314, the training module 164 may train a machine-learning model using the correlation data from the correlation database 156 and the parameter data from the parameter database 154. Training a machine-learning model may comprise the steps of providing a set of parameters and/or correlation data to the machine-learning model and receiving at least one prediction from the machine-learning model, where the prediction comprises a value of a parameter for which data is not provided to the machine-learning model. The prediction value is then compared against a known value, and a correction factor is applied based upon the variance of the prediction from the known value. This process may be repeated multiple times for a predetermined number of iterations or until the accuracy of the model is within an acceptable level. In some models, such as using generative adversarial networks, known values may be similarly determined via mathematical simulation.

Additionally, the machine-learning models may be integrated into third party or proprietary mathematical models that simulate the process in part or in its entirety by measuring real-time data and computing one or more operating scenarios that may improve the efficiency of an algae reactor 116. An example is the identification of insufficient and excessive fluid circulation zones that inhibit microalgal cultivation. A mathematical model may predict the impact of different parameter values, such as gas or liquid injection or withdrawals at various points within the algae reactor 116, comparing the predicting impact, to identify the optimal parameter values. The trained machine-learning model may be sent to the control system 158.

At step 316, the control system 158 may poll one or more sensors measuring at least one parameter related to the collection and/or processing of CO₂, the operation of an algae reactor 116, and/or the harvest and processing of algae. Parameters relating to the collection and/or processing of CO₂ may include those relevant to one or more of a gas chiller 104, gas dryer 106, gas compressor 108, gas filter 110, and/or gas scrubber 112. Relevant parameters may include concentrations of CO₂ and/or one or more component gases received from a gas source 102 and/or at any point during processing of the CO₂ containing gas. Additional relevant parameters may include moisture content, pH, temperature, pressure, etc. Parameters relating to the operation of an algae reactor 116 may include any of energy usage, light intensity and/or penetration, CO₂ concentration in solution, concentration of one or more nutrients such as nitrogen, potassium, phosphates, etc.

Additional relevant parameters may include the concentration of algae, pH, temperature, agitation and/or movement of algae resulting from an agitator 130 or the lifting action from a bubble generating device 124, etc. Parameters relating to the harvesting of algae may include the water content of harvested algae, weight, temperature, etc. In an embodiment, measuring a concentration of CO₂ from a gas source 102 at 15% and the moisture content at 14%. In another embodiment, measuring a temperature within an algae reactor 116 of 110.8° F. and a pH of 7. CO₂ fed photobioreactor control system 100. In an embodiment, the sensor 152 data is received in real time. Real time may relate to the continuous acquisition of data or may refer to minimizing the acquisition and processing time of one or more sensors 152. Real time may instead refer to a sensor 152 sample interval, such as once per second.

At step 318, the control system 158 may receive a prediction for the future value of one or more parameters based upon the data received from one or more sensors 152 using the trained machine-learning model. For example, the machine-learning model may predict that the algae in the algae reactor 116 will reach 112° F. within the next 5 minutes. As another example, the machine-learning model may predict that the pH of an algal slurry in the algae reactor 116 will reach 7.0 within the next hour.

At decision block 320, the control system 158 may determine whether action is required based upon the prediction received from the machine-learning model. In an embodiment, the prediction requires action if the predicted value for one or more parameters will exceed the operational parameters for the algae reactor 116. For example, if the maximum tolerable temperature for algae is 111° F., and the predicted future temperature is 112° F., then action is required to cool or otherwise prevent heating of the algae reactor 116 to avoid exceeding the maximum operational temperature. This may be done by reducing heat generation, such as by turning off or pulsing the light source 122. Alternatively, water which is cooler than the temperature of the algal slurry may be added to the algae reactor 116. In another embodiment, the future pH is expected to be 7.0. If the operational range for pH is between 6.5 and 7.5, then no action is required.

At step 322, the control system 158 may perform the required action, if necessary, based upon the future parameter value predicted by the machine-learning model. In an embodiment, the maximum tolerable temperature for algae is 111° F. and the predicted future temperature is 112° F., therefore action is required to cool the temperature of the algal slurry within the algae reactor. The action of adding cooled water at a temperature of 65° F. is taken. In some cases, reports may be generated. The reports may with respect to performance, operating history, learned correlations, and actions recommended or taken.

At step 324, the control system 158 may send a control status of the algae reactor 116 to the control system 158. The control status may indicate one or more actions taken, such as cooling the temperature of an algal slurry, adjusting the pH, increasing the amount of light, beginning harvest of the algae, etc. In other embodiments, the control status may comprise a general status such as nominal operation, or anomalous operation, and/or may indicate whether an action is necessary or in progress.

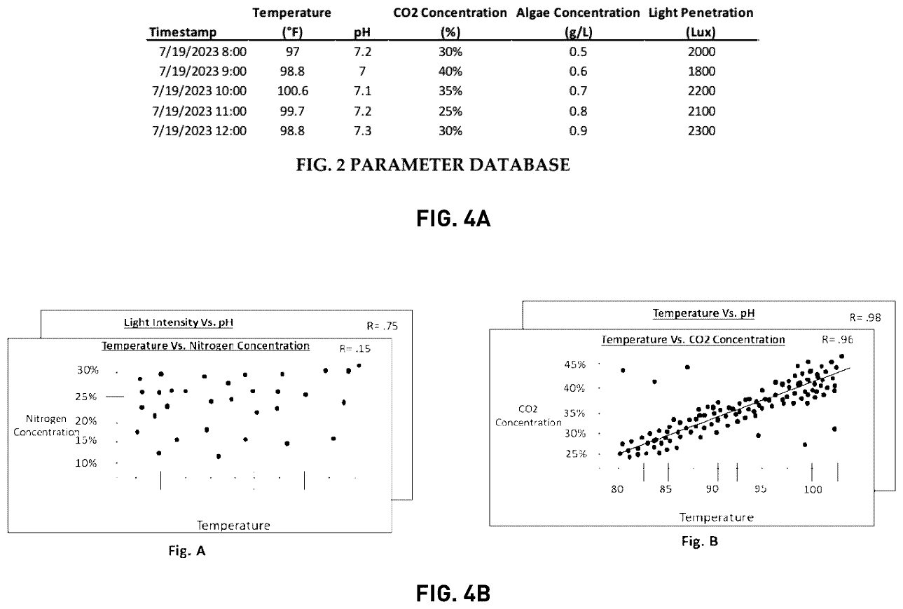

FIG. 4A illustrates an example parameter database 154.

The parameter database 154 may store data collected by one or more sensors 152 measuring one or more parameters related to the collection and/or processing of gasses containing CO₂, the operation of an algae reactor 116 such as the control and optimization of internal environmental conditions which may include the introduction, distribution, and circulation and concentration of gasses including CO₂, oxygen, and other gases in air, process gases, and in solution within the water used to cultivate microalgae, the movement and circulation of fluids and solids including the multiplication and growth of microalgae, light management to optimize algae cultivation and minimize energy consumption, the amount and type of nutrient feed, algae contamination detection systems.

Examples of parameters may include any of the concentration of CO₂ and/or one or more component gases in a CO₂ containing gas from a gas source 102 and/or at any point during processing of the CO₂ containing gas. Additional parameters may include moisture content, pH, temperature, pressure, etc. Parameters relating to the operation of an algae reactor 116 may include any of energy usage, light intensity and/or penetration, CO₂ concentration in solution, concentration of one or more nutrients such as nitrogen, potassium, phosphates, etc. Additional relevant parameters may include the concentration of algae, pH, temperature, agitation and/or movement of algae resulting from an agitator 130 or the lifting action from a bubble generating device 124, etc.

Parameters relating to the harvesting of algae may include the water content of harvested algae, weight, temperature, etc. The parameters may be used to monitor, analyze, and control one or more systems related to the algae reactor 116 to optimize algae production and quality while minimizing energy consumption, and resource usage including water and nutrients. Methods of analyzing and controlling an algae reactor 116 may comprise one or more algorithms including machine-learning and may likewise apply to continuous or batch modes of operation. The parameter database 154 is populated by the data collection module 160 and is used by the correlation module 162, training module 164, and may additionally be used and populated by the control module 166. The parameter database 154 may be stored in a cloud communication network 140, on a third-party network 142, or on a monitoring and monitoring system 146. An example parameter database 154 is shown below.

FIG. 4B illustrates an example correlation database 156.

The correlation database 156 may store correlation data generated by the correlation module 162 comprising at least a first parameter, a second parameter, and a correlation coefficient representing the relationship between the first parameter and the second parameter such that the higher the correlation coefficient, the higher the relationship between the first and second parameters. The correlation database 156 is used by the training module 164 and may further be used by the control module 166.

FIG. A illustrates a first parameter and a second parameter with a low correlation coefficient, illustrating no relationship or a weak relationship between the first parameter and the second parameter. FIG. B illustrates a first parameter and a second parameter with a high correlation coefficient, illustrating a strong relationship between the first parameter and the second parameter.

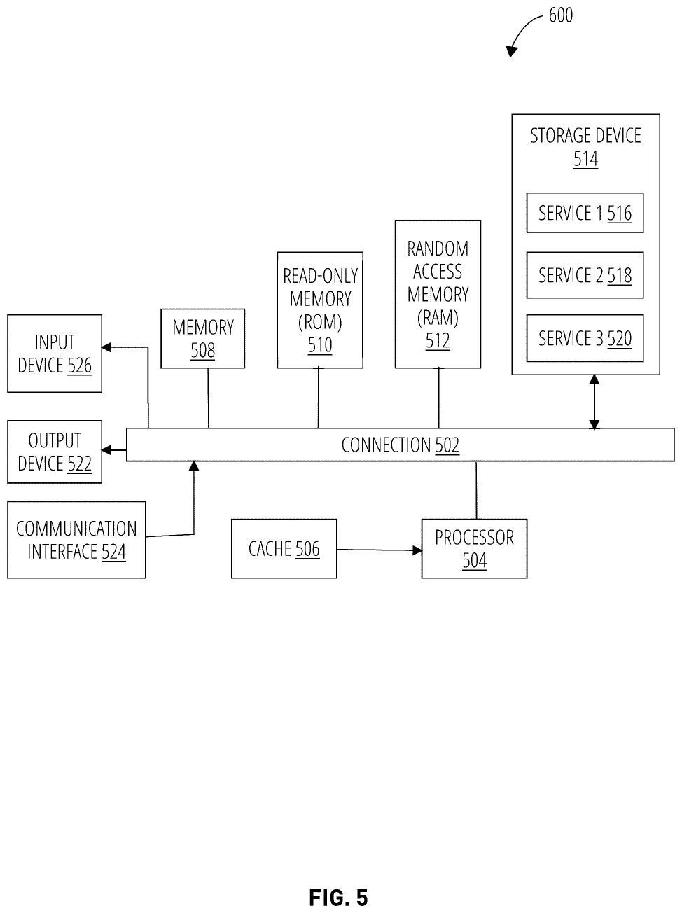

FIG. 5 illustrates a block diagram of an exemplary computing system that may be used to implement an embodiment of the present invention. The example of computer system 500 can be for example any computing device making up CO₂ fed photobioreactor control system 100, or any component thereof in which the components of the system are in communication with each other using connection 502. Connection 502 can be a physical connection via a bus, or a direct connection into processor 504, such as in a chipset architecture. Connection 502 can also be a virtual connection, networked connection, or logical connection.

In some embodiments, computing computer system 500 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example computing computer system 500 includes at least one processing unit (CPU or processor) 504 and connection 502 that couples various system components including system memory 508, such as read-only memory (ROM) 510 and random-access memory (RAM) 512 to processor 504. Computing system 500 can include a cache of high-speed memory 508 connected directly with, in close proximity to, or integrated as part of processor 504.

Processor 504 can include any general purpose processor and a hardware service or software service, such as services 516, 518, and 520 stored in storage devices 514, configured to control processor 504 as well as a special-purpose processor where software instructions are incorporated into the actual processor design. Processor 504 may essentially be a completely self-contained computing system, containing multiple cores or processors, a bus, memory controller, cache, etc. A multi-core processor may be symmetric or asymmetric.

To enable user interaction, computing computer system 500 includes an input device 526, which can represent any number of input mechanisms, such as a microphone for speech, a touch-sensitive screen for gesture or graphical input, keyboard, mouse, motion input, speech, etc. Computing system 500 can also include output device 522, which can be one or more of a number of output mechanisms known to those of skill in the art. In some instances, multimodal systems can enable a user to provide multiple types of input/output to communicate with computer system 500. Computing system 500 can include communication interface 524, which can generally govern and manage the user input and system output. There is no restriction on operating on any particular hardware arrangement, and therefore the basic features here may easily be substituted for improved hardware or firmware arrangements as they are developed.

Storage device 514 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 514 can include software services, servers, services, etc., that when the code that defines such software is executed by the processor 504, it causes the system to perform a function. In some embodiments, a hardware service that performs a particular function can include the software component stored in a computer-readable medium in connection with the hardware components, such as processor 504, connection 502, output device 522, etc., to carry out the function.

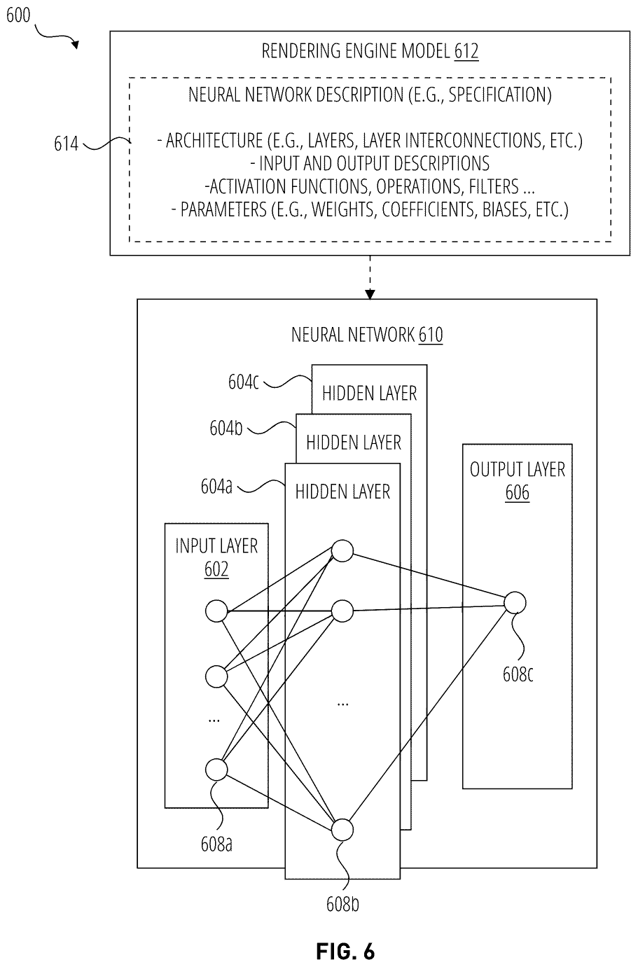

FIG. 6 illustrates an example neural network architecture.

Architecture 600 includes a neural network 610 defined by an example neural network description 614 in rendering engine model (neural controller) 612. The neural network 610 can represent a neural network implementation of a rendering engine for rendering media data. The neural network description 614 can include a full specification of the neural network 610, including the neural network architecture 600. For example, the neural network description 614 can include a description or specification of the architecture 600 of the neural network 610 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

The neural network 610 reflects the architecture 600 defined in the input layer 602. In this example, the neural network 610 includes an input layer 602, which includes input data, such as one or more correlations and the selected parameter data. In one illustrative example, the input layer 602 can include data representing a portion of the input media data such as a patch of data corresponding to the input data. The neural network 610 includes hidden layers 604a through 604n (collectively “604” hereinafter). The hidden layers 604 can include n number of hidden layers, where n is an integer greater than or equal to one. The number of hidden layers can include as many layers as needed for a desired processing outcome and/or rendering intent.

The neural network 610 further includes an output layer 606 that provides an output (e.g., predicted future parameters values) resulting from the processing performed by the hidden layers 604. In one illustrative example, the output layer 606 can provide predicted future parameters values. The neural network 610 in this example is a multi-layer neural network of interconnected nodes. Each node can represent a piece of information. Information associated with the nodes is shared among the different layers and each layer retains information as information is processed. In some cases, the neural network 610 can include a feed-forward neural network, in which case there are no feedback connections where outputs of the neural network are fed back into itself. In other cases, the neural network 610 can include a recurrent neural network, which can have loops that allow information to be carried across nodes while reading in input. Information can be exchanged between nodes through node-to-node interconnections between the various layers.

Nodes of the input layer 602 can activate a set of nodes in the first hidden layer 604a. For example, as shown, each of the input nodes of the input layer 602 is connected to each of the nodes of the first hidden layer 604a. The nodes of the hidden layers hidden layer 604a can transform the information of each input node by applying activation functions to the information. The information derived from the transformation can then be passed to and can activate the nodes of the next hidden layer (e.g., 604b), which can perform their own designated functions. Example functions include convolutional, up-sampling, data transformation, pooling, and/or any other suitable functions. The output of the hidden layer (e.g., 604b) can then activate nodes of the next hidden layer (e.g., 604n), and so on. The output of the last hidden layer can activate one or more nodes of the output layer 606, at which point an output is provided. In some cases, while nodes (e.g., nodes 608a, 608b, 608c) in the neural network 610 are shown as having multiple output lines, a node has a single output and all lines shown as being output from a node represent the same output value. In some cases, each node or interconnection between nodes can have a weight that is a set of parameters derived from training the neural network 610.

For example, an interconnection between nodes can represent a piece of information learned about the interconnected nodes. The interconnection can have a numeric weight that can be tuned (e.g., based on a training dataset), allowing the neural network 610 to be adaptive to inputs and able to learn as more data is processed. The neural network 610 can be pre-trained to process the features from the data in the input layer 602 using the different hidden layers 604 in order to provide the output through the output layer 606. In an example in which the neural network 610 is used to identify predicted future parameters values, the neural network 610 can be trained using training data that includes features from the one or more correlations and the selected parameter data. For instance, training data can be input into the neural network 610, which can be processed by the neural network 610 to generate outputs which can be used to tune one or more aspects of the neural network 610, such as weights, biases, etc. In some cases, the neural network 610 can adjust weights of nodes using a training process called backpropagation. Backpropagation can include a forward pass, a loss function, a backward pass, and a weight update. The forward pass, loss function, backward pass, and parameter update is performed for one training iteration.

The process can be repeated for a certain number of iterations for each set of training media data until the weights of the layers are accurately tuned. For a first training iteration for the neural network 610, the output can include values that do not give preference to any particular class due to the weights being randomly selected at initialization. For example, if the output is a vector with probabilities for predicted future parameters values, the probability value for each of the predicted future parameter values may be equal or at least very similar. With the initial weights, the neural network 610 is unable to determine low level features and thus cannot make an accurate determination of what the classification of the object might be. A loss function can be used to analyze errors in the output. Any suitable loss function definition can be used. The loss (or error) can be high for the first training dataset (e.g., features from the one or more correlations and the selected parameter data) since the actual values will be different than the predicted output.

The goal of training is to minimize the amount of loss so that the predicted output comports with a target or ideal output. The neural network 610 can perform a backward pass by determining which inputs (weights) most contributed to the loss of the neural network 610 and can adjust the weights so that the loss decreases and is eventually minimized. A derivative of the loss with respect to the weights can be computed to determine the weights that contributed most to the loss of the neural network 610. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 610 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for down-sampling), and fully connected layers. In other examples, the neural network 610 can represent any other neural or deep learning network, such as an autoencoder, a deep belief nets (DBNs), a recurrent neural networks (RNNs), etc.

The functions performed in the processes and methods may be implemented in differing orders. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. A computer-implemented method for controlling an algae reactor, the computer-implemented method comprising:
storing correlation data in a correlation database;
determining that one or more correlations between a set of the correlation data is above a predetermined correlation threshold, wherein the set includes two or more parameters and one or more correlation coefficients;
querying a parameter database to select parameter data associated with the two or more parameters;
training a machine-learning model based on the one or more correlations and the selected parameter data to predict future values of the two or more parameters;
polling one or more sensors associated with the algae reactor for sensor data associated with the two or more parameters;
predicting, by the trained machine-learning model, future parameter values of the two or more parameters based on the polled sensor data;
determining that action is required based on the predicted future parameter values exceeding an operational parameter threshold; and
instructing the algae reactor to perform the required action.

2. The computer-implemented method of claim 1, wherein the sensors measure at least the two or more parameters, and further comprising:
initializing the sensors for an operation of the algae reactor that is related to collection or processing of CO₂; and
establishing communication with the sensors by sending a message from a controller and receiving a response indicating that the message was received and that the sensors are operating properly.

3. The computer-implemented method of claim 2, further comprising calibrating the sensors by comparing measurements from two or more similar sensors to known parameters, wherein calibration provides a correction factor to compensate for any identified deviations in the measurements.

4. The computer-implemented method of claim 1, wherein the sensors measure at least the two or more parameters, and further comprising:
polling the sensors regarding an operation of the algae reactor that is related to collection or processing of CO₂; and
storing collected data from the polled sensors in the parameter database.

5. The computer-implemented method of claim 1, further comprising:
querying third-party databases for data related to growth characteristics associated with a respective algae growing the algae reactor or data related to manufacturer information related to components of the algae reactor; and
calibrating the predetermined correlation threshold based on the data from the third-party databases.

6. The computer-implemented method of claim 1, further comprising:
for each of the parameters:
selecting the parameter;
selecting a different parameter;
calculating a respective correlation coefficient; and
storing the respective correlation coefficient for the parameter and the different parameter.

7. The computer-implemented method of claim 1, wherein training the machine-learning model includes:
extracting features from the one or more correlations and the selected parameter data; and
training the machine-learning model based on the extracted features with a loss function, wherein the loss function optimizes parameters of the machine-learning model to minimize errors between predicted future parameters values and actual values.

8. The computer-implemented method of claim 1, further comprising sending a report that includes a status update indicating one or more of actions taken, an overall operational state of the algae reactor, performance, operating history, learned correlations, and recommendations.

9. The computer-implemented method of claim 1, further comprising selecting at least a first parameter, a second parameter, and a correlation coefficient from the correlation data.

10. A photobioreactor control system comprising:
a memory storing correlation data in a correlation database; and
one or more processors executing instructions, wherein the processor execute the instructions to:
determine that one or more correlations between a set of the correlation data is above a predetermined correlation threshold, wherein the set includes two or more parameters and one or more correlation coefficients;
query a parameter database to select parameter data associated with the two or more parameters;
train a machine-learning model based on the one or more correlations and the selected parameter data to predict future values of the two or more parameters;
poll one or more sensors associated with the algae reactor for sensor data associated with the two or more parameters;
predict, by the trained machine-learning model, future parameter values of the two or more parameters based on the polled sensor data;
determine that action is required based on the predicted future parameter values exceeding an operational parameter threshold; and
instruct an algae reactor of the photobioreactor control system to perform the required action.

11. The photobioreactor control system of claim 10, wherein the sensors measure at least the two or more parameters, and wherein the processors execute further instructions to:
initialize the sensors for an operation of the algae reactor that is related to collection or processing of CO₂; and
establish communication with the sensors by sending a message from a controller and receiving a response indicating that the message was received and that the sensors are operating properly.

12. The photobioreactor control system of claim 11, wherein the processors execute further instructions to calibrate the sensors by comparing measurements from two or more similar sensors to known parameters, wherein calibration provides a correction factor to compensate for any identified deviations in the measurements.

13. The photobioreactor control system of claim 10, wherein the sensors measure at least the two or more parameters, and the processors execute further instructions to:
poll the sensors regarding an operation of the algae reactor that is related to collection or processing of CO₂; and
store collected data from the polled sensors in the parameter database.

14. The photobioreactor control system of claim 10, wherein the processors execute further instructions to:
query third-party databases for data related to growth characteristics associated with a respective algae growing the algae reactor or data related to manufacturer information related to components of the algae reactor; and
calibrate the predetermined correlation threshold based on the data from the third-party databases.

15. The photobioreactor control system of claim 10, wherein the processors execute further instructions to:
for each of the parameters:
select the parameter;
select a different parameter;
calculate a respective correlation coefficient; and
store the respective correlation coefficient for the parameter and the different parameter.

16. The photobioreactor control system of claim 10, wherein the processors train the machine-learning model by:
extracting features from the one or more correlations and the selected parameter data; and
training the machine-learning model based on the extracted features with a loss function, wherein the loss function optimizes parameters of the machine-learning model to minimize errors between predicted future parameters values and actual values.

17. The photobioreactor control system of claim 10, further comprising a communication interface that communicates over a communication network, wherein the communication interface sends a status update indicating any actions taken or an overall operational state of the algae reactor.

18. The photobioreactor control system of claim 10, wherein the processors execute further instructions to select at least a first parameter, a second parameter, and a correlation coefficient from the correlation data.

19. A non-transitory computer-readable storage medium having instructions embodied thereon, the instructions executable by a computer to perform a method for controlling an algae reactor, the method comprising:
storing correlation data in a correlation database;
determining that one or more correlations between a set of the correlation data is above a predetermined correlation threshold, wherein the set includes two or more parameters and one or more correlation coefficients;
querying a parameter database to select parameter data associated with the two or more parameters;
training a machine-learning model based on the one or more correlations and the selected parameter data to predict future values of the two or more parameters;
polling one or more sensors associated with the algae reactor for sensor data associated with the two or more parameters;
predicting, by the trained machine-learning model, future parameter values of the two or more parameters based on the polled sensor data;
determining that action is required based on the predicted future parameter values exceeding an operational parameter threshold; and
instructing the algae reactor to perform the required action.
